(19) United States
(12) Patent Application Publication
(10) Pub. No.: US 2004/0029210 A1
(43) Pub. Date: Feb. 12, 2004

(54) Assay for paralytic shellfish toxin

(76) Inventors: Cedric Emile Francois Robillot, Tomsville (AU); Lyndon Edwin Llewellyn, Wulguru (AU); James Nigel Burnell, Wulguru (AU)

Correspondence Address: MOORE & VAN ALLEN, PLLC, 2200 W MAIN STREET, SUITE 800, DURHAM, NC, 27705, US

(21) Appl. No.: 10/450,351

(22) PCT Filed: Dec. 12, 2001
(86) PCT No.: PCT/AU01/01605

(30) Foreign Application Priority Data
Dec. 12, 2000 (AU) PR 2034
Aug. 20, 2001 (AU) PR 7145

(51) Int. Cl. C12Q 1/02

(52) U.S. Cl. 435/29

(57) ABSTRACT

A method of detecting and/or measuring the amount of a paralytic shellfish toxin (PST) present in a sample, comprising the steps of: 1) providing an isolated and purified saxiphilin, or fragment thereof which contains a saxitoxin binding site; 2) contacting it with the sample; 3) mearsuring binding of PST contained in the sample to said isolated and purified saxiphilin; and correlating the amount of binding with either the presence or absence of PSTs in the sample or with the PST concentration in the sample.

### TECHNICAL FIELD

This invention relates to the isolation and purification of a saxitoxin-binding polypeptide, saxiphilin, and to methods, assays and devices for the detection, concentration, purification and extraction of saxitoxin which employ purified saxiphilin. In particular the invention relates to an economical, robust, high throughput assay which does not require the use of radioactively-labelled reagents, and which is suitable for use in the field.

### BACKGROUND OF THE INVENTION

Paralytic shellfish poisoning caused by ingestion of fish, crustaceans or molluscs containing toxins derived from dinoflagellates is a world-wide problem resulting in severe human illness, which often results in death. The poisoning is caused by paralytic shellfish toxins (PSTs) which are the family of toxins related to the archetypal molecule saxitoxin (STX). In addition, blooms of toxic freshwater algae can contaminate water supplies with the same neurotoxins that cause paralytic shellfish poisoning. This toxin contaminated water can have dire consequences for humans, livestock and wildlife.

The general structure of PSTs is as follows:

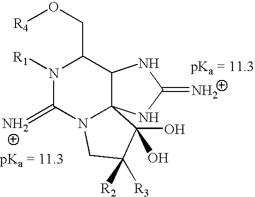

| | R₁ | R₂ | R₃ | R₄ |
|---|---|---|---|---|
| STX | H | H | H | CONH₂ |
| dcSTX | H | H | H | H |
| B1 | H | H | H | CONHSO₃⁻ |
| B2 | OH | H | H | CONHSO₃⁻ |
| C1 | H | H | OSO₃⁻ | CONHSO₃⁻ |
| C2 | H | OSO₃⁻ | H | CONHSO₃⁻ |
| C3 | OH | H | OSO₃⁻ | C CONHSO₃⁻ |
| C4 | OH | OSO₃⁻ | H | CONHSO₃⁻ |
| neoSTX | OH | H | H | CONH₂ |
| dcNeoSTX | OH | H | H | H |
| GTX2 | H | H | OSO₃⁻ | CONH₂ |
| GTX3 | H | OSO₃⁻ | H | CONH₂ |
| GTX1 | OH | H | H | CONH₂ |
| GTX4 | OH | OSO₃⁻ | H | CONH₂ |

This family of toxins can be divided into three broad categories: the saxitoxins, which are highly potent neurotoxins, and which are not sulphated; the gonyautoxins (GTXs), which are singly sulphated; and the N-sulphocarbamoyl-11-hydrosulphate C-toxins, which are less toxic than the STXs or GTXs.

The toxicity of the PSTs is a result of their binding to voltage-dependent sodium channels, which blocks the influx of sodium ions, and thus blocks neuromuscular transmission. This causes respiratory paralysis, for which no treatment is available. In some outbreaks of paralytic shellfish poisoning up to 40% of the victims have died. The PSTs bind to the same site on the sodium channel as tetrodotoxins, which have a completely different structure (Hall et al., 1990). In some cases, tetrodotoxins can occur together with PSTs, and therefore any assay for detection of PSTs must be able to distinguish them from tetrodotoxins.

The dinoflagellates which are the source of PSTs periodically form algal blooms, known as red tides (Anderson, 1994). Molluscs, fish, and crustaceans, including species of commercial significance or which are raised using aquaculture techniques, may feed on these dinoflagellates and accumulate the toxins. It is not possible to detect by gross examination whether an individual marine animal contains the toxin, and therefore there is a risk that humans will inadvertently consume toxin-containing animals. It is therefore necessary to monitor species which are to be consumed for the presence of PSTs, in order to avoid the risk of poisoning and to prevent social and economic cost.

More than 20 natural analogues of saxitoxin are known, and their toxicity to mammals varies. Some of the naturally-occurring PSTs are listed in Table 1.

TABLE 1
Some of the naturally occurring PSTs

| | Common | | CAS |
|---|---|---|---|
| | literature | | Registry |
| Trivial name | abbreviations | Systematic name | number |
| Saxitoxin | STX | 1H,10H-pyrrolo [1,2-c]purine-10,10-diol-2,6-diamino-4[[(aminocarbonyl)oxy]methyl]-3a,4,8,9- | 35523-89-8 |
| | | terrahydro, [3aS-(3aα, 4α, 10aR*)] | |
| α-saxitoxinol | — | 1H,8H-pyrrolo[1,2-c]purine-4-methanol,2,6-diamino-3a,4,9,10-tetrahydro-10-hydroxy-,α- | 75420-34-7 |
| | | carbamate, [3aS-(3aα, 4α, 10β, 10aR*)] | |
| β-saxitoxinol | — | 1H,8H-pyrrolo[1,2-c]purine-4-methanol,2,6-diamino-3a,4,9,10-tetrahydro-10-hydroxy-,α- | 75352-30-6 |
| | | carbamate, [3aS-(3aα, 4α, 10α, 10aR*)] | |
| Neosaxitoxin | neoSTX | 1H,10H-pyrrolo[1,2-c]purine-10,10-diol, 2-amino-4-[[(aminocarbonyl)oxy]methyl]- | 64296-20-4 |
| | | 3a,4,5,6,8,9-hexahydro-5-hydroxy-6-imino, [3aS-(3aα, 4α, 10aR*)] | |
| Gonyautoxin I | GTX I or | 1H,10H-pyrrolo[1,2-c]purine-9,10,10-triol-2-amino-4-[[(aminocarbonyl)oxy]methyl]- | 60748-39-2 |
| | GTX₁ | 3a,4,5,6,8,9-hexahydro-5-hydroxy-6-imino-9-(hydrogen sulfate), [3aS-(3aα, 4α, 9β, 10aR*)] | |
| Gonyautoxin II | GTX II or | 1H,10H-pyrrolo[1,2-c]purine-9,10,10-triol-2,6-diamino-4-[[(aminocarbonyl)oxy]methyl]- | 60508-89-6 |
| | GTX₂ | 3a,4,8,9-tetrahydro-9-(hydrogen sulfate), [3aS-(3aα, 4α, 9β, 10aR*)] | |
| Gonyautoxin III | GTX III or | 1H,10H-pyrrolo[1,2-c]purine-9,10,10-triol-2,6-diamino-4-[[(aminocarbonyl)oxy]methyl]- | 60537-65-7 |
| | GTX₃ | 3a,4,8,9-tetrahydro-9-(hydrogen sulfate), [3aS-(3aα, 4α, 9α, 10aR*)] | |
| Gonyautoxin IV | GTX IV or | 1H,10H-pyrrolo[1,2-c]purine-9,10,10-triol-2-amino-4-[[(aminocarbonyl)oxyl]methyl] | 64296-26-0 |
| | GTX₄ | 3a,4,5,6,8,9-hexahydro-5-hydroxy-6-imino-9-(hydrogen sulfate), [3aS-(3aα, 4α, 9α, 10aR*)] | |
| Gonyautoxin V | GTX V, GTX₅ | Carbamic acid, sulfo-, C-[2,6-diamino-3a,4,9,10-tetrahydro-10,10-dihydroxy-1H,8H- | 64296-25-9 |
| | or B1 | pyrrolo[1,2-c]purin-4-yl)methyl]ester, [3aS-3aα, 4α, 10aR*)] | |
| Gonyautoxin VI | GTX VI, GTX₆ | Carbamic acid, sulfo-, C-[(2-amino-3a,4,5,6,9,10-hexahydro-5,10,10-trihydroxy-6-imino-1H, | 82810-44-4 |
| | or B2 | 8H-pyrrolo[1,2-c]purine-4-yl)methyl]ester, [3aS-(3aα, 4α, 10aR*)] | |
| Gonyautoxin VIII | GTX VIII or | Carbamic acid, sulfo-, C-[[2,6-diamino-3a,4,9,10-tetrahydro-10,10-dihydroxy-9-(sulfoxy)- | 80226-62-6 |
| | GTX₈ or C2 | 1H,8H-pyrrolo[1,2-c]purine-4-yl]methyl]ester, [3aS-(3aα, 4α, 9α, 10aR*)] | |
| epi-gonyautoxin VIII | epi-GTX VIII | Carbamic acid, sulfo-, C-[[2,6-diamino-3a,4,9,10-tetrahydro-10,10-dihydroxy-9-(sulfoxy)- | 80173-30-4 |
| | or C1 | 1H,8H-pyrrolo[1,2-c]purine-4-yl]methyl]ester, [3aS-(3aα, 4α, 9β, 10aR*)] | |

The incidence of algal blooms appears to be increasing world-wide, possibly as a result of increased eutrophication of coastal waters and global warming, and consequently the incidence of outbreaks of paralytic shellfish poisoning or of contamination of shellfish or other organisms with PSTs is also increasing. For example, in 2000 alone, four people in Sabah, Malaysia, were poisoned and shellfisheries were closed for four months. Shellfisheries in Manila Bay, the Philippines, were closed for several months; nine people were poisoned and five admitted to hospital in Washington State, and shellfisheries were closed for several months in the year 2000 in Cape Cod and South Maine, both in the United States; all shellfishing on the west coast of the North Island of New Zealand was stopped in May 2000 as the result of an algal bloom, which was approaching the green-lipped mussel beds, which produce mussels worth NZ$84 million annually ; in Scotland shellfishing was banned in June, 2000; and blooms leading to instances of paralytic shellfish poisoning have occurred in South Africa and China; as the result of contamination detected in July, 2000 in Canada, 3000 aquaculture salmon were destroyed. In particular, in the United States, approximately 150 outbreaks of contamination of shellfish have occurred in the last decade, with closures of shellfisheries of up to twelve months resulting; closures of three years have occurred in some parts of Scotland; in Morocco, in 1994, four people died and 74 were admitted to hospital; almost 1600 people have been poisoned in the Philippines since 1983, whereas virtually no such incidence were observed before 1983; and in one outbreak in India in 1997, seven people died, 500 were admitted to hospital, and the ban on shellfishing resulted in the loss of jobs for 1000 families.

Unfortunately, although the need for a simple, robust and reliable method of detecting contamination of marine organisms to be used for human consumption is evident, methods which are currently available are not satisfactory. Regular testing of shellfish to ensure that toxic product does not enter the market place is required (Van Egmond and Dekker, 1995). Currently, the only officially endorsed method is the mouse lethality bioassay approved by the Association of Official Analytical Chemists (AOAC) official methods of analysis, section 959.08 E,. 1990. This requires intraperitoneal injection of mice with an HCl extract of potentially toxic organisms such as shellfish, and observation of the time from injection to death (Sommer and Meyer, 1937; Hungerford, 1995). The mice must come from a colony of mice which is regularly standardised for its sensitivity to reference toxin samples, and the sample must be diluted so that death occurs between 5 and 7 minutes. The assay is inhumane, expensive, and unpopular, and is at risk of being prohibited as a result of animal welfare regulation, particularly in countries such as the European Union, the Netherlands and Germany. Of even greater concern is that the mouse bioassay assay has a sensitivity of only 180 μg STX/1 (Johnson and Mulberry, 1966).

This lack of sensitivity means that there is a serious risk that levels of PSTs sufficient to cause toxicity in humans may not be detected. For example, children in the Philippines have died as the result of ingestion of shellfish when mouse lethality bioassays indicated that shellfish contained only 40 μg STX/100 g shellfish meat, which equates to around 200 μg when takes into account dilution due to extraction solvent plus shellfish. This level of toxicity is the same as the detection limit for the mouse lethality bioassay.

This problem has led to attempts to develop alternative assays, based on

(a) detecting the presence of intoxicating organisms by biological observation,

(b) in situ detection using methods such as DNA probes, or

(c) detecting the presence of toxins in the marine organism by biochemical, physiological or chemical assay.

One approach utilises blockage of the voltage-gated sodium channel (VGSC), a large transmembrane protein in excitable cells which allows passage of ions through a central pore when it opens in response to alterations in cellular potential difference. (See for example Doucette et al., 1997; Jellett et al., 1992; Vieytes et al., 1993). These-assays are radioligand assays (Weigele and Barchi, 1978), which can be adapted to a microtitre plate format which increases the sample throughput (Doucette et al., 1997). Alternatively cultured cells hyperstimulated so as to increase ion flow through the sodium channel may be used (Jellett et al., 1992; U.S. Pat. No. 5,420,011 and U.S. Pat. No. 5,858,687).

However, these assays are expensive and technically complex, requiring either radioactively-labelled reagents or cell cultures. Moreover they are sensitive to pH fluctuations, because at pH greater than 6.7 PSTs are readily displaced from the ion channel, are similarly sensitive to cation concentration, and, more importantly, are non-specific because they also detect tetrodotoxin. None of these assays is suitable for field use. Chemical assays are complicated by the fact that the individual toxins are tremendously variable in structure, ranging from very polar to lipophilic, and from low to high molecular weight. Furthermore, these chemical methods require the use of standard samples of the known toxins, and any new and biologically active PSTs will not be measurable by these methods. Thus assays based on detection using antibodies or using chemical methods such as high performance liquid chromatography, mass spectrometry, or capillary electrophoresis may not detect the broad range of toxins.

A simple, rapid preliminary clean-up method for crude shellfish extracts, coupled with a bench or desktop lateral flow immuno-chromatographic assay marketed by Jellett Biotek, enables a preliminary result to be obtained within ten minutes; however, confirmatory screening using liquid chromatography-mass spectrometry is required. The preliminary clean-up uses ammonium formate mobile phase on a 5 cm solid-phase column suitable for lipophilic toxins, and this is followed by LCMS on a Tosoh-Haas amide 40 column using a 60-90% gradient of tetranitrile-2 mM ammonium formate, pH3.5. A preliminary report was presented by M. A. Quilliam at the International Marine Biotechnology Conference, Townsville, September 2000.

We have utilised a different approach, which relies on a receptor protein known as saxiphilin, which is completely unrelated to the VGSC in either amino acid sequence or of functional properties, and which specifically binds STX but not tetrodotoxins (Llewellyn and Moczydlowski, 1994). The ability of saxiphilin to bind STX has been used in a low-throughput radioligand binding assay for detection of PSTs in blue-green algae, crustaceans and molluscs (Carmichael et al., 1997; Negri and Llewellyn, 1998). This utilises displacement of ³H-labelled STX from saxiphilin. We have utilised a crude saxiphilin-containing extract to develop a microtitre plate assay for detection of PSTs (Llewellyn et al., 1998; Llewellyn and Doyle, 2000). While this assay provides high throughput, sensitivity and accuracy, and has the advantage that it does not suffer from interference-by other compounds present in shellfish extracts or from the acidic pH necessary to maintain stability of toxin during extraction from shellfish, it still suffers from the disadvantage that it requires radioactively-labelled material.

The saxiphilin utilised in the assay is a crude preparation prepared by homogenising specimens of the centipede Ethmostigmus rubripes in buffer containing a protease inhibitor cocktail. While this preparation provides good sensitivity, there is still a problem in availability of the reagent, and the fact that it is not a defined, reproducible preparation. Therefore there is still a need in the art for a rapid, robust assay which is suitable for field use, for example on fishing vessels, or at aquaculture facilities, and which detects a wide range of STXs.

It will be clearly understood that, although a number of prior art publications are referred to herein, this reference does not constitute an admission that any of these documents form part of the common general knowledge in the art, in Australia or in any other country.

### SUMMARY OF THE INVENTION

According to one aspect of the present invention there is provided a method of detecting and/or measuring the amount of paralytic shellfish toxin (PST) present in a sample, comprising the steps of:

1) providing an isolated and purified invertebrate saxiphilin, or a fragment thereof which contains a saxitoxin binding site;

2) contacting it with the sample;

3) measuring binding of PSTs to the invertebrate saxiphilin; and

correlating the amount of binding with either the presence or absence of PSTs in the sample or with the PST concentration in the sample.

The invertebrate saxiphilin, or fragment thereof, may be coupled to a detectable label or immobilised on a solid support. The detectable label may be any suitable label, as would be understood by the person skilled in the art and may be coupled to a solid support in any convenient manner.

In a further aspect of the present invention there is provided method of measuring the amount of paralytic shellfish toxin (PST) present in a sample, comprising the steps of:

(a) pre-treating the filters of a microtitre filtration plate with a polycation;

(b) adding to wells of the plate a known amount of a labelled saxiphilin comprising an isolated and purified invertebrate saxiphilin, or a fragment thereof which contains a saxitoxin binding site labelled with a detectable marker, and a series of dilutions of material suspected to comprise paralytic shellfish toxin;

(c) incubating the plate for a time sufficient to permit binding of any paralytic shellfish toxin present to the labelled saxiphilin;

(d) aspirating the contents of each well through the filter of the well to remove components other than labelled saxiphilin and compounds bound thereto;

(e) rinsing each well and filter to remove residual unbound compounds; and

(f) measuring the amount of labelled saxiphilin retained by the filter,

in which the degree of binding of labelled saxiphilin when compared with a control sample indicates the amount of paralytic shellfish toxin present in the sample.

In a further aspect of the present invention there is provided an isolated and purified invertebrate saxiphilin coupled to a solid support.

In a still further aspect of the present invention there is provided an isolated purified invertebrate saxiphilin labelled with a detectable label.

In a further aspect, the invention provides a method of isolation of an invertebrate saxiphilin, comprising the steps of:

(a) homogenising individuals of a saxiphilin-producing arthropod species in a physiological buffer comprising protease inhibitors;

(b) subjecting the homogenate to low-speed centrifugation to remove cell debris;

(c) subjecting the supernatant from step (b) to high-speed centrifugation; and

(d) precipitating saxiphilin from the supernatant by exposure to ammonium sulphate;

(e) redissolving the precipitate at pH 5.0-6.5 and centrifuging to remove non-saxiphilin molecules;

(f) exposing the supernatant from (e) to a matrix to which saxiphilin binds, such as a glass fibre-polyethylene imine (PEI) support matrix; and

(g) eluting bound material from the matrix under high salt conditions.

Advantageously the saxiphilin is precipitated by exposure to 40-60% ammonium sulphate. Prior to this step, the pH may be temporarily reduced to 5.0 to precipitate some of the non-saxiphilin.

In step (g), the saxiphilin is typically eluted by NaCl or KCl at a concentration from 600 mM to saturation, in buffer at pH5-9. A number of different buffer systems may be used.

Optionally, further purification may be obtained, for example by chromatofocussing on PBE 94 resin quilibrated with 25 mM imidazole-HCl pH 7.4 and eluting with a solution containing 25 mL Polybuffer 74, brought to a final volume of 200 mL and a pH of 4.0 with HCl.

Polybuffer removal and buffer exchange can then be achieved by size exclusion chromatography or desalting on a column, such as PD-10 columns from Amersham Pharmacia Biotech.

The arthropod species may be any species which produces saxiphilin. See for example Llewellyn et al., 1997. Preferably the arthropod is a centipede, such as Ethmostigmus rubripes, an isopod, such as an Oniscus species, a spider, such as Araneus. c.f. Cavaticus, a Xanthid crab, or an insect of the family Clopterygidae.

More preferably the arthropod is a centipede, most preferably Ethmostigmus rubripes. Saxiphilin from this species has been shown to be able to bind PSTs of all the structural sub-class of the PST family with comparable affinity. The arthropod may conveniently be anaesthetised by exposure to hypothermia. Homogenisation can be carried out using any convenient apparatus, such as a Heidolph tissue homogeniser. One suitable homogenisation buffer is 20 mM HEPES-NaOH, pH7.4, containing 0.5 mM EDTA 1 μM leupeptin, 1 μM pepstatin, 0.5 μM aprotonin, and 1 μM phenylmethylsulphonyl fluoride. Suitably 2 ml buffer is used per gram of arthropod material. The low speed centrifugation may conveniently be performed at 8000 g for 10 minutes, followed by high speed centrifugation at 50,000 g for 20 minutes. The supernatant following high-speed centritugation may be frozen in liquid nitrogen and stored at 80° C. prior to further processing.

The PEI support matrix is prepared by conventional methods, for example by incubation of glass fibre with 0.3% PEI in water solution (v/v) for at least 1 hour and removal of the PEI by draining or aspiration under vacuum.

The isolated saxiphilin may be used for detection of PSTs, using the microtitre plate assay which we have previously described (Llewellyn and Doyle 2000; Lewellyn et al., 1998), utilising saxiphilin labelled with a non-radioactive label. The person skilled in the art will be aware of suitable labels, which include fluorescent and chemiluminescent labels, colloidal gold, latex microbeads, liposome-encapsulated dyes and enzymic labels, although these are not favoured as the enhancement of the signal is time dependent due to the need for an enzymatic reaction to take place. The liposome encapsulated dyes may be biotinylated or tagged in some other way to facilitate their capture in an assay. Suitable detection methods using each of these labels are known in the art.

The isolated saxiphilin of the invention is also useful in preparation of affinity materials for purification, concentration or extraction of PSTS, for example in testing water quality of waters suspected to be contaminated by algal blooms. For example, the isolated saxiphilin may be coupled to a suitable solid support, which may then be packed in a column or a cartridge. In one preferred embodiment, the solid support is packed in a cartridge adapted for attachment to a syringe. The person skilled in the art will be aware of suitable coupling methods and supports, for example cyanogen bromide-activated matrices such as agarose; epoxy activated matrices; glutaraldehyde-activated silica; carboxymethylcellulose hydrazide; polyacrylamide hydrazide and oxirane acrylic beads. PSTs can be eluted from the affinity material by treatment with a small volume (eg 1-5 ml) of acid, urea or concentrated salts.

For assays being performed in the laboratory, this preliminary purification may be performed prior to assay of a sample of material suspected to be contaminated with PSTs.

Material suitable for use in the assay or the preliminary concentration method of the invention can be a tissue extract, for example from vertebrates such as fish or a mammalian species who may have ingested PST contaminated material; invertebrates such as molluscs, including shellfish or cephalopods; macroscopic algae such as seaweed; microalgae including cyanobacteria, dinoflagellates and the like; or bacteria. Biological fluids such as blood, urine or saliva of patients suspected to be suffering from PST poisoning, or water samples, such as drinking water supplies suspected of contamination or water from regions manifesting algal blooms, which may contain dissolved toxins released by the bloom organisms, can also be tested. In addition, samples containing synthetic PSTs can be utilised.

PSTs can be extracted from tissue to be tested using any suitable aqueous or alcoholic solvent; preferably the solvent is at acid pH, since saxitoxin is susceptible to degradation under basic conditions. Optionally the extraction may be performed at elevated temperature. A particularly suitable solvent is that utilised in the method endorsed by the Association of Official Analytical Chemists, namely 0.1 N HCl.

There are various specific methodologies for carrying out assays of the invention, and various preferred embodiments of the invention are described below.

In one embodiment the invention provides a method of measuring the amount of a paralytic shellfish toxin present in a sample, comprising the steps of

(a) pre-treating the filters of a microtitre filtration plate with a polycation;

(b) adding to wells of the plate a known amount of invertebrate saxiphilin labelled with a detectable marker, and a series of dilutions of material suspected to comprise paralytic shellfish toxin;

(c) incubating the plate for a time sufficient to permit binding of the paralytic shellfish toxin to the saxiphilin;

(d) aspirating the contents of each well through the filter of the well to remove components other than saxiphilin and compounds bound thereto;

(e) rinsing each well and filter to remove residual unbound compounds; and

(f) measuring the amount of labelled saxiphilin retained by the filter,

in which the degree of binding of labelled saxiphilin when compared with a control sample indicates the amount of paralytic shellfish toxin present in the sample.

Preferably in step (b) the sample comprises a buffer to maintain pH in the range 6.5 to 9, and optionally also comprises a chloride salt, such as sodium chloride or potassium chloride, present at a concentration up to 500 mM. Typically the total volume present in the well is 50 to 350 μl, preferably 100 to 200 μl, more preferably 150 μl. In step (c) the incubation is carried out at 0 to 30° C., preferably at room temperature, for at least 30 minutes; the incubation is preferably for 60 to 120 minutes, more preferably 90 minutes, but can be continued up to about 8 hours. In step (e), the rinse may be performed using any suitable solution, such as a solution buffered at the same pH as for step (b). A single rinse will usually be adequate; however, each well is typically rinsed 2 to 3 times.

In a preferred embodiment, the protocol uses a total volume of 150 μl containing 20 mM MOPS-NaOH (pH 7.4), 200 mM NaCl, and 1 nM labelled STX centipede saxiphilin according to the invention and incubation at room temperature (−25° C.) for 90 min prior to aspiration through the filters. Wells are rinsed three times with 180 μl ice-cold water. The optimum amount of saxiphilin may readily be determined by routine experimentation.

In a further aspect, the invention provides a kit for measuring the amount of paralytic shellfish toxin in a sample, comprising

(a) a microtiter plate;

(b) saxiphilin according to the invention, labelled with a detectable marker;

(c) extraction buffer for extracting material to be tested from a sample of an organism or tissue to be tested; and optionally

(d) a concentrating means for concentrating paralytic shellfish poisons in the extract or removal of contaminants that may interfere with the assay.

Preferably the concentrating means is a column or cartridge comprising a solid support material coupled to purified saxiphilin according to the invention.

According to a still further aspect of the present invention there is provided a device for measuring the amount of paralytic shellfish toxin (PST) present in a sample, comprising:

an immobilised invertebrate saxiphilin, or a fragment thereof which contains a saxitoxin binding site;

means for introducing a sample to said immobilised sample to said immobilised saxiphilin, or fragment thereof; and

means for correlating the amount of binding with either the presence or absence of PSTs or with PST concentration in the sample.

According to a still further aspect of the present invention there is provided a device for measuring the amount of paralytic shellfish toxin (PST) present in a sample, comprising:

an immobilised PST;

means for introducing a sample to said immobilised PST;

means for introducing a predetermined amount of an isolated and purified invertebrate saxiphilin to the sample;

means for measuring binding of the invertebrate saxiphilin introduced to said immobilised PST; and

means for correlating competition for binding between the immobilised PST and any PST contained in the sample with PST concentration in the sample.

Typically an invertebrate saxitoxin is used and this has advantageously been purified as described above.

The device may be a biosensor, and therefore include means for translating the binding event into an electronic signal.

Advantageously, this is by a detection of the change of mass of the protein upon binding. It will therefore be appreciated that, since saxiphilin is a relatively large protein, enhancements in the sensitivity of detection may be achieved through using fragments of the saxiphilin protein in place of immobilised saxiphilin, provided that they contain the saxitoxin binding site. If a fragment is used it will be appreciated that the change in mass upon binding is greater as a proportion of the total weight of the system. In a competitive binding assay in which a PST is immobilised it is preferable to employ full length saxiphilin, as the reverse is true.

According to a still further aspect of the invention there is provided a method for the concentration, purification and/or extraction of paralytic shellfish toxins (PSTs), comprising the steps of:

providing an immobilised invertebrate saxiphilin, or a fragment thereof which contains a saxitoxin binding site;

contacting a sample suspected of containing a PST with said immobilised saxiphilin for a sufficient time for the PST to bind the immobilised saxiphilin; and

optionally, eluting the bound PST from the immobilised saxiphilin.

This method may be used, among other things, to detoxify shellfish and purify water.

There is also provided the use of isolated saxiphilin in the preparation of affinity materials for concentration, purification and/or extraction of paralytic shellfish toxins.

There is also provided an affinity material for concentration, purification and/or extraction of paralytic shellfish toxins, comprising an isolated and purified invertebrate saxiphilin, or fragment thereof which contains a saxitoxin binding site coupled to a solid support.

Advantageously the solid support is selected from the group consisting of azolactone matrices, cyanogen bromide-activated matrices; epoxy activated matrices; glutaraldehyde-activated silica; carboxymethylcellulose hydrazide; polyacrylamide hydrazide and oxirane acrylic beads.

For the purposes of this specification it will be clearly understood that the word “comprising” means “including but not limited to”, and that the word “comprises” has a corresponding meaning.

### BRIEF DESCRIPTION OF THE FIGURES

FIG. 1 is a schematic representation of views from above and from the side of a diagnostic test strip for detecting the presence of PSTs.

FIG. 2 is a schematic representation of an alternative diagnostic test strip;

FIG. 3 is a schematic representation illustrating the principle of a microtitre plate assay for PSTs;

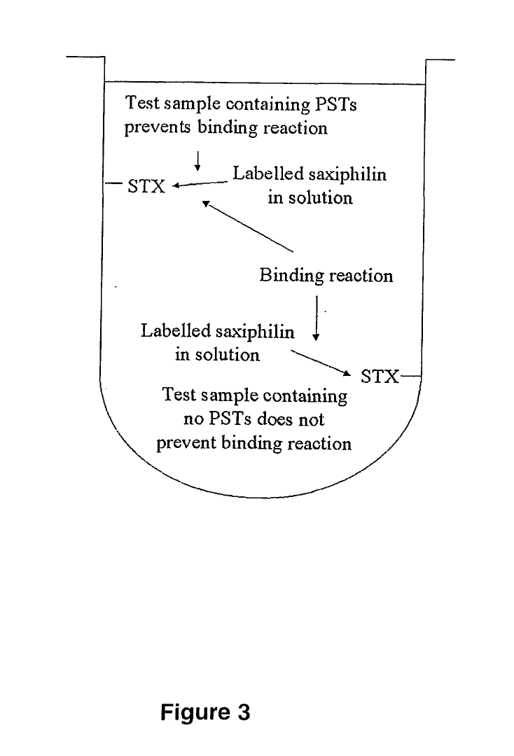

FIG. 4 shows schematically the competitive binding in a surface-plasmon resonance (SPR) sensor;

FIG. 5 is a schematic representation of a saxiphilin-based surface-plasmon resonance (SPR) sensor for the rapid quantification of PSTs;

FIG. 6 is a graph showing the eluted radioactivity of the binding experiments from Example 3;

FIG. 7 is a bar graph showing specific binding of radioactivity in pH 5.0 peak in FIG. 6; and

FIG. 8 is a graph showing the elution profile in the stability testing described in Example 3.

### DETAILED DESCRIPTION OF THE INVENTION

The invention will now be described in detail by way of reference only to the following non-limiting examples and drawings.

### EXAMPLE 1

Purification of Saxiphilin

Crude saxiphilin was obtained by homogenising specimens of the centipede Ethmostigmus rubripes in 10 mM Tris-HCl, 0.2 mM EDTA (pH 7.4) (2×10 sec bursts with a Waring blender at maximum setting; 3 ml buffer:1 g centipede) containing a cocktail of protease inhibitors (5 mM EDTA, 1 μM pepstatin, 1 μM aprotonin, 100 μM phenylmethylsulfonyl fluoride). After centrifuging the homogenate at 24,000 g for 20 min, the pellet was rehomogenised and centrifuged as above. The two supernatants were combined and passed through a 0.2 μm cellulose acetate filter (Nalgene). The saxiphilin was then precipitated from this supernatant by exposure to 40-60% ammonium sulphate, followed by removal of non-saxiphilin molecules from this precipate by redissolving into a buffered solution of Ph 5.0-6.5 and centrifuging to leave a supernatant containing saxiphilin.

This supernatant was then exposed to a glass fibre-polyethylene imine (PEI) support matrix, prepared by incubation of glass fibre with 0.3% PEI in water solution (v/v) for at least 1 hour and removal of the PEI by draining or aspiration under vacuum. Saxiphilin was eluted from the matrix using high salt, with the saxiphilin typically being eluted by NaCl or KCl at a concentration from 600 mM to saturation, at pH 5-9.

The protein has also been subjected to chromatofocussing on PBE 94 resin equilibrated with 25 mM imidazole-HCl pH 7.4 and eluting with a solution containing 25 mL Polybuffer 74, brought to a final volume of 200 mL and a pH of 4.0 with HCl. Polybuffer removal and buffer exchange can then be achieved by size exclusion chromatography or desalting columns, such as PD-10 columns from Amersham Pharmacia Biotech.

### EXAMPLE 2

Use of Purified Saxiphilin in Assay

a) Diagnostic Test Strips

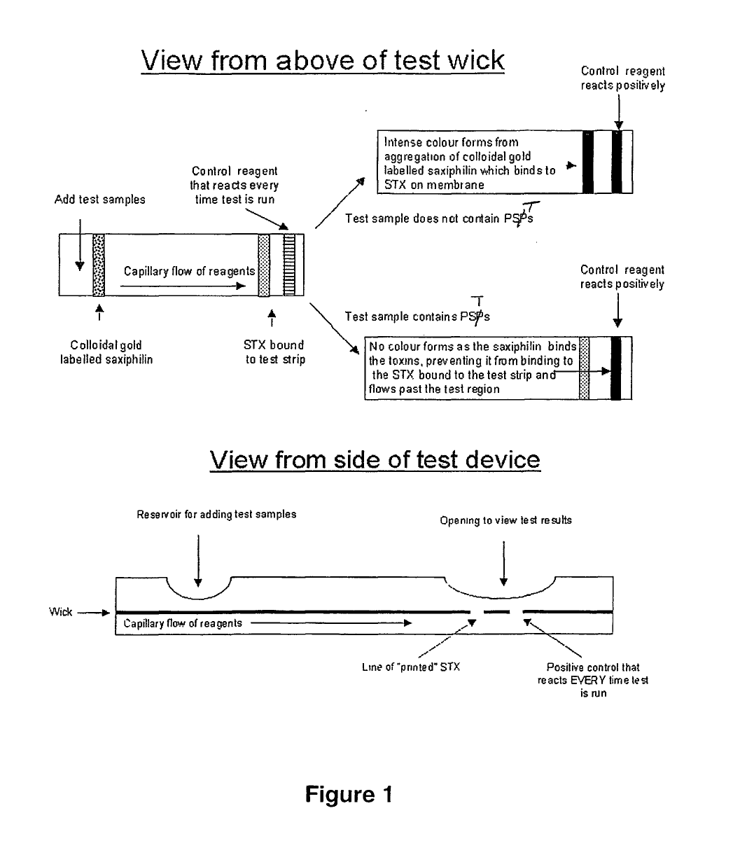

One diagnostic kit for qualitative detection of PSTs using the purified saxiphilin of the invention is in the form of a test strip. The kit uses a solid matrix, or “wick”, upon which the reaction occurs. The kit has a band of saxitoxin at one end of this solid matrix, applied using a method known as “printing”. This immobilises the saxitoxin, which then acts as an anchor for modified saxiphilin (described below) as it flows past the “printed” saxitoxin. If the modified saxiphilin is already bound to a PST from a test sample, then it will be unable to bind to “printed” saxitoxin, and will continue to flow, preventing colour development. If the test sample has no PSTs, then the modified saxiphilin will bind to the band of “printed” saxitoxin, forming a coloured spot. To generate a coloured spot upon anchorage of saxiphilin, saxiphilin is conjugated to colloidal gold or coloured latex microbeads. The principle is that the colloidal gold, an intensely coloured reagent, is aggregated into a spot obvious to the human eye when the conjugated saxiphilin binds the STX immobilised on to the membrane. As it flows past the band of printed saxitoxin, it will stop and aggregate, or continue and not form a band visible to the human eye. This is illustrated schematically in FIG. 1. Thus this form of assay provides a qualitative “yes/no” assay for the presence of PSTs in a sample. The test strip provides a positive control.

An alternative approach is to use a liposome encapsulated-dye. Liposomes provide instantaneous enhancement, and have considerable potential for automated assays.

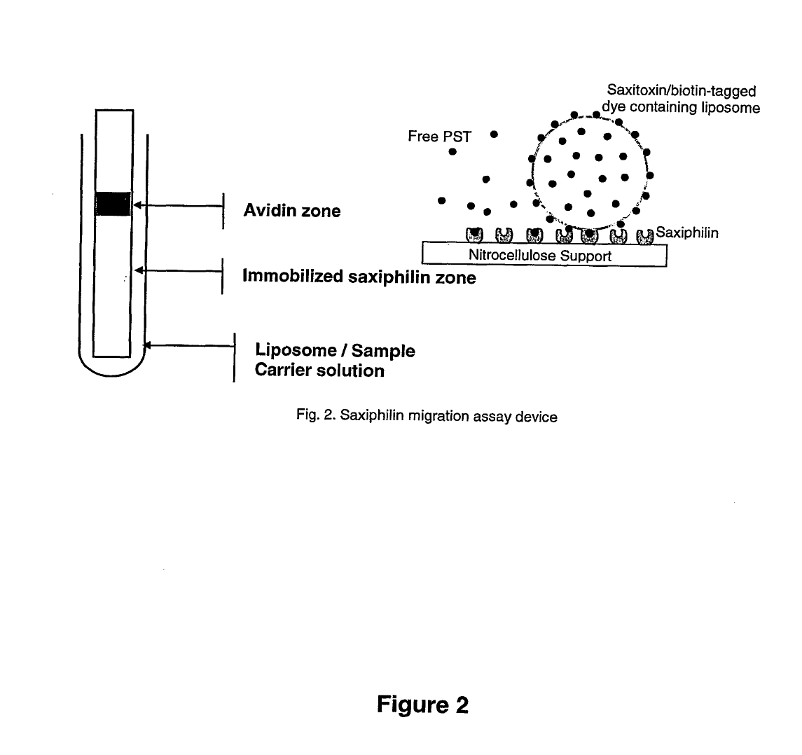

The experimental system is a competitive receptor assay and consists of a wicking reagent containing saxitoxin/biotin-tagged liposomes with entrapped dye and a plastic-backed nitrocellulose strip that has an immobilized saxiphilin competition zone and a liposome avidin capture zone in an ascending sequence (FIG. 2). A mixture of the wicking reagent and a sample containing an unknown quantity of PSTs is allowed to migrate along the strip by capillary action. In the saxiphilin zone, competitive binding with the PSTs receptor occurs. The unbound liposomes, proportional to the amount of saxitoxin in the sample, are carried into the liposome capture zone where they are concentrated. The color intensity of the saxiphilin zone and the avidin zone are estimated either visually or by scanning densitometry.

The amount of immobilized saxiphilin must be as low as possible to increase sensitivity to PSTs, but sufficient to allow visual detection of liposomes.

A typical migration assay requires approximately 100 μL sample solution and should reach the ppb detection level in less than 10 minutes, corresponding to PSTs detection limits in the low ng range. Liposomes are highly stable molecules that can be stored at least one year at +4° C. and several months at room temperature. This assay would be easily used in to field testing, without any special equipment or technical skills required. The kit would include special holders for the individual strips, in which openings are provided for sample application and optical readout. This low cost saxiphilin migration sensor allows easy and rapid screening of environmental samples and constitute an unparalleled and reliable tool for the PSTs risk assessment.

(b) Microtitre Plate Assay

Establishing the assay in a microtitre plate format allows its more sophisticated use, and enables quantitative results to be obtained. One preferred format is a binding inhibition asay. Saxitoxin is coated on a 96-well microtitre plate. Test samples are mixed with labelled saxiphilin and added to the 96 well plate. Toxin-free samples do not prevent the labelled saxiphilin from binding to the saxitoxin-coated surface of the wells of the 96 well plate, forming a coloured region. Toxin-containing samples inhibit colour formation with the degree of inhibition being proportional to the amount of toxin present. The plate is then read in a spectrophotometic plate reader and the amount of toxin quantified.

A further possible technique for quantification of PSTs involves high-performance liquid chromatography coupled to a post-column oxidation system and a fluorescence detector. This procedure is complex and requires expensive PSTs standards. However, the combination of highly specific saxiphilin-based identification and sensitive detection by means of surface plasmon resonance (SPR), overcomes the drawbacks related to chromatographic techniques.

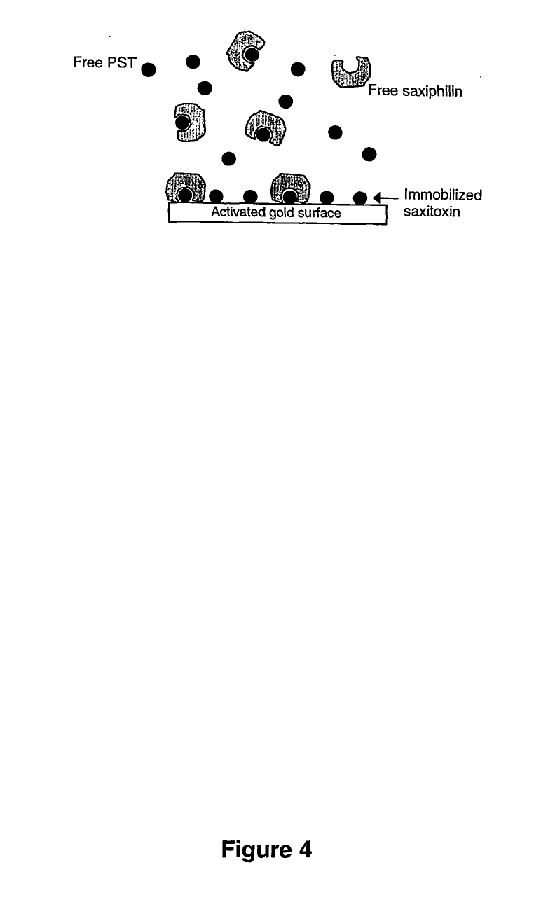

The device requires a PST such as saxitoxin to be coupled to an activated gold surface, which is exposed to a liquid sample during the analysis. The SPR sensor detects changes in the reflection of laser light caused by the change of refractive index at the metal-liquid interface. Thus, when saxitoxin is coupled to the activated gold surface of the sensor, a refractive index alteration is induced by saxiphilin binding (FIG. 4). In the case where PSTs are present in the sample, a competition occurs between the free PSTs and bound saxitoxin, and the resulting signal decrease can be quantified.

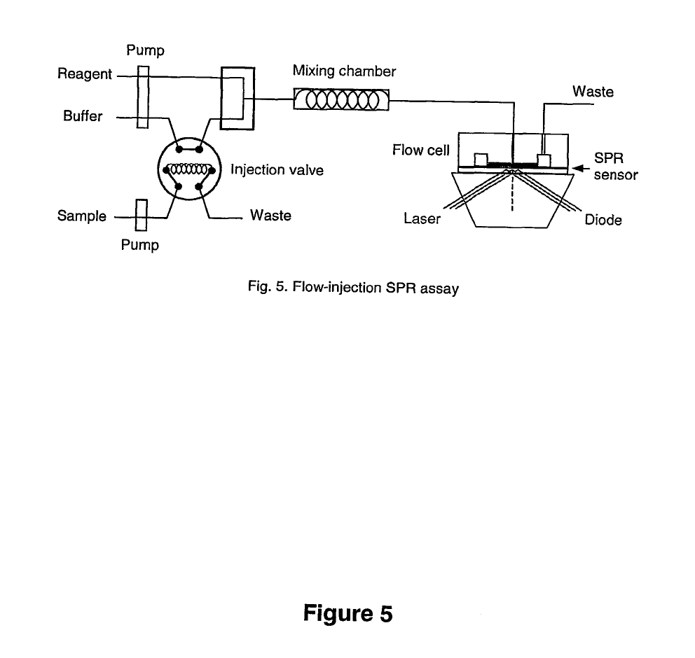

This flow-injection receptor assay consists of i) reagent pumping and sample injection systems, ii) a mixing cell where the competitive receptor assay occurs and iii) the SPR sensor including a flow cell and optical devices (FIG. 5). This system can be fully automated, such as in the BIACORE 2000™ available from Biacore AB.

### EXAMPLE 3

Affinity Column Preparation

By linking saxiphilin to a solid phase, its ability to bind saxitoxin may be used to separate saxitoxin from liquid samples passed over the saxiphilin linked solid support. Since binding to centipede saxiphilin is pH dependent, the bound toxin can then be eluted.

Making the Saxiphilin Affinity Column

Isolated saxiphilin was used to prepare an affinity column using the Ultralink™ Tm kit manufactured by Pierce Chemical Company. This resin relies upon azolactone coupling chemistry and uses an inert semi-rigid resin with medium to fast flow characteristics.

The method used was as follows:

1. The ammonium sulphate precipitated saxiphilin was resuspended into the Pierce supplied coupling buffer of BupH citrate-carbonate buffer (pH 9.).

2. This resuspended saxiphilin was added to 0.15 g of 3M Emphaze Biosupport medium AB 1 (supplied by Pierce) which hydrates the resin and binds available proteins. The resin swelled to 1 ml.

3. After 1 hour of gentle mixing, the resin and saxiphilin preparation was packed into a mini-column, and the resin was allowed to settle

4. The column was then washed with phosphate buffered saline (15 mls)

5. 4 mls of quench buffer (3M ethanolamine pH 9.0) was then added and the resin gently mixed in this buffer for 2.5 hours

6. The column was then washed with 15 ml phosphate buffered saline

7. The top of the resin was then sealed with a porous disc insert

8. Wash the column with 15 ml 1M NaCl

9. Wash the column with 15 ml 100 mM HEPES-NaOH (pH 7.4) and it is ready for testing for ability to bind saxitoxin

Testing for Column Binding of Saxitoxin

Tritiated saxitoxin (Amersham Pharmacia Biotech) was used to measure the columns ability to bind saxitoxin.

Three 2 μl aliquot of the ³H-STX (60 nM) was counted in a scintillation counter to measure how much radioactivity was going to be applied to the column. These aliquots contained 2113±42 counts per minute (cpm).

200 μl of ³H-STX (=211,300 cpm—see point above) was added to the column and allowed to flow into the resin. The ³H-STX is prepared by 150-fold dilution of the commercially supplied ³H-STX (in 0.01 M acteic acid containing 2% ethanol) into 1 mM citrate buffer (pH 5.0). The column was then washed with 5 ml 100 mM HEPES-NaOH (pH 7.4). The flow through was collected. The column was then washed successively with 5 ml of the following with each sample collected separately:

2nd wash 100 mM HEPES-NaOH (pH 7.4)

3rd wash 100 mM HEPES-NaOH (pH 7.4)

100 mM HEPES-NaOH (pH 6.0)

100 mM HEPES-NaOH (pH 5.0)

0.001N HCl

0.005N HCl

0.01 N HCl

0.05 N HCl

0.1 N HCl

0.5 N HCl

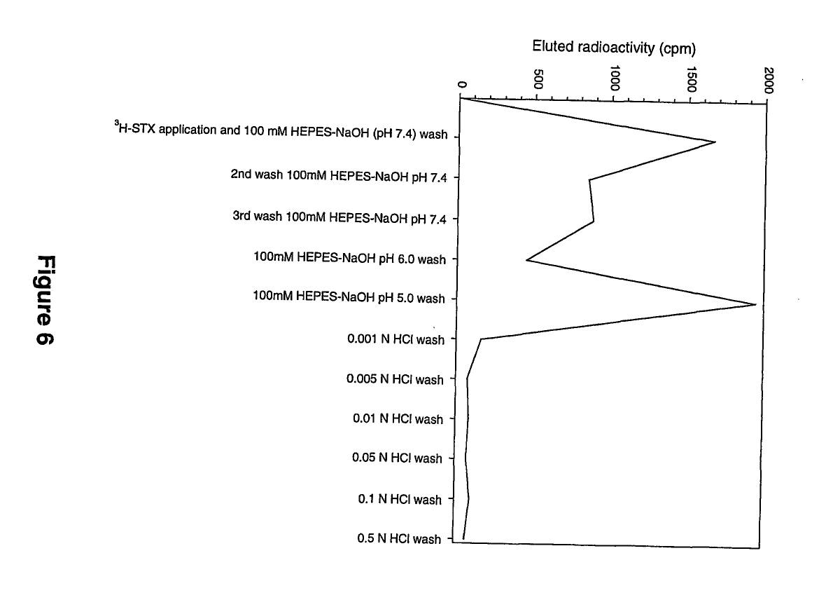

The eluted radioactivity is depicted in FIG. 6.

As can be seen, there are two major peaks of radioactivity. The first elutes in the first fraction and is essentially unbound by the column. The second peak is eluted by 100 mM HEPES-NaOH pH 5.0. Tritiated saxitoxin contains free tritium and so these two peaks were tested for biological activity by measuring their ability to bind to the two known receptors for saxitoxin, namely the sodium channel and saxiphilin.

Measuring Biological Activity of Eluted Peaks form Saxiphilin Column

Conditions in the Assay Used Were:

Sodium channel: 100 mM MOPS-NaOH (pH 7.4), 100 mM choline chloride, 100 μl of fractions 1 and 5 (wash and first pH 7.4 wash, pH 5.0 wash respectively), 10 μl rat brain vesicles containing sodium channels in a final volume of 250 μl. Samples were done in duplicate and a negative control containing an excessive amount of unlabelled tetrodotoxin was also performed to define specific levels of any bound ³H-STX.

Saxiphilin: 100 mM MOPS-NaOH (pH 7.4), 100 mM sodium chloride, 100 μl of fractions 1 and 5 (wash and first pH 7.4 wash, pH 5.0 wash respectively), 1.5 μl characterised centipede saxiphilin preparation in a final volume of 250 μl. Samples were done in duplicate and a negative control containing an excessive amount of unlabelled saxitoxin was also performed to define specific levels of any bound ³H-STX.

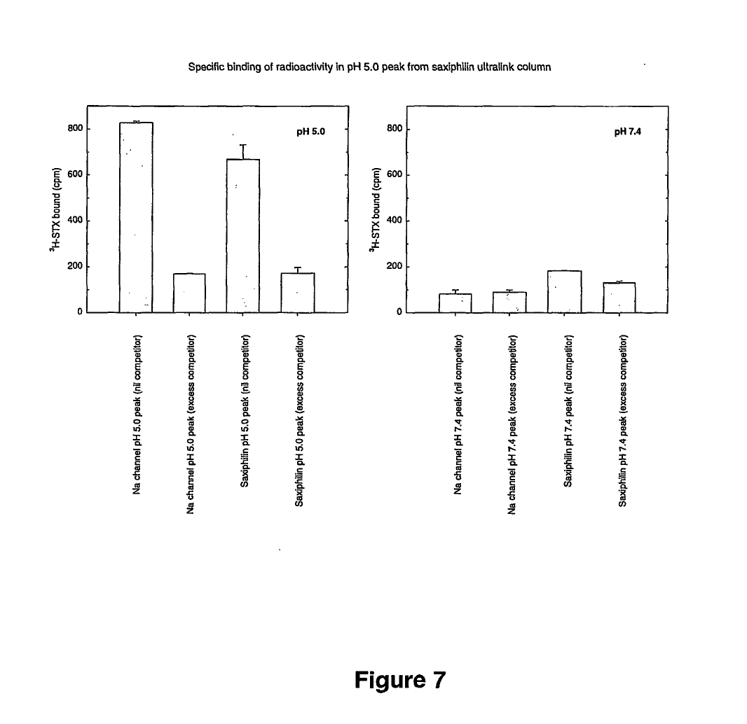

As can be seen in FIG. 7, only the pH 5.0 eluted peak of radioactivity from the column retained biological activity ie it could bind to the two known saxitoxin receptors. The pH 7.4 peak did not contain any such biological activity (except for a minimal amount of receptor binding activity in the saxiphilin assay) indicating that the radioactivity was tritium unincorporated into saxitoxin (a known property of commercially supplied ³H-STX).

Stability of Column After Initial Treatment

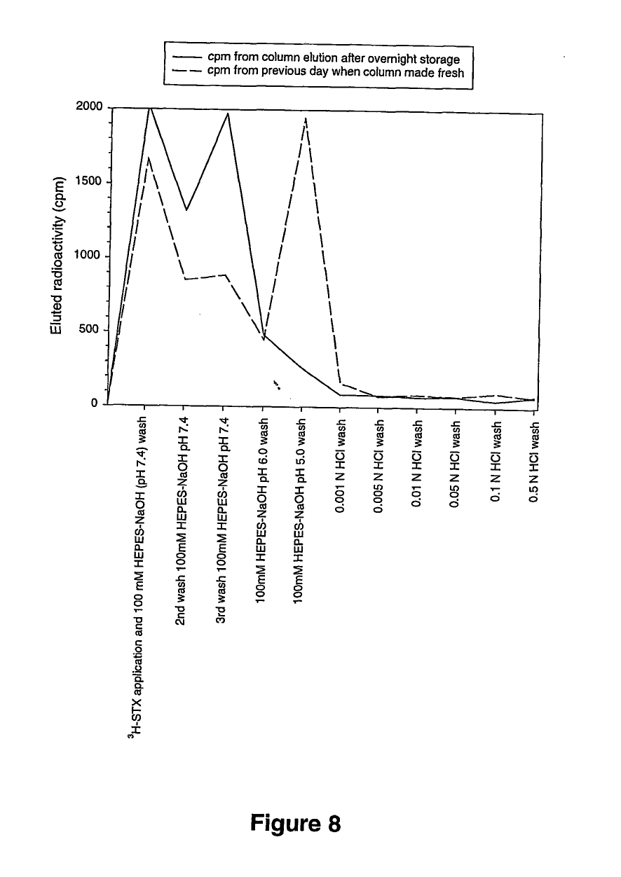

After the final 0.5N HCl wash, the column was washed with 20 ml 100 mM HEPES-NaOH (pH 7.4) and stored overnight at 4° C. This column was removed and allowed to return to room temperature and the above elution experiment was repeated and the profile shown in FIG. 8 was obtained.

As can be seen, the second peak of activity has shifted to be eluted by the 3rd wash with HEPES-NaOH (pH 7.4) which may indicate degradation of the saxiphilin. These peaks were not tested for biological activity.

Thus it will be appreciated that saxiphilin can be bound to a solid phase chromatography resin such as Pierce's Emphaze Biosupport medium AB 1. On this column saxitoxin is bound by the saxiphilin and separated from other material (eg free tritium), then the saxitoxin can be eluted from the column with pH 5.0. The eluted saxitoxin retains biological activity. Treatment with acid (eg 0.5 N HCl) may degrade the linked saxiphilin. Non-specific retention of the ³H-STX by the treated resin is not apparent.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

References

Anderson D. M. (1994) Red Tides. Sci. Am. 271, 62-68.

Carmichael W. W., Evans W. R., Yin Q. Q., Bell P and Moczydlowski E. (1997) Evidence for paralytic shellfish poisons in the freshwater cyanobacterium Lyngbya wollei(Fallow ex Gomont) comb. nov. Appl. Environ. Micro. 63, 3104-3110.

Doucette G. J., Logan M. M., Ramsdell J. S. and Van Dolah F. M. (1997) Development and preliminary validation of a microtiter plate-based receptor binding assay for paralytic shellfish poison toxins. Toxicon 35, 625-636.

Fernandez, M. L. and Cembella, A. D., 1995, Mammalian bioassays. Manual on Harmful Marine Microalgae, edited by G. M. Hallegraeff, D. M. Anderson, and A. D. Cembella (Paris: UNESCO), pp 213-228.

Hall S., Strichartz G., Moczydlowski E., Ravindran A., Reichardt P. B. (1990) The saxitoxins. Sources, Chemistry and Pharmacology. In: Marine Toxins: Origin, Structure and Molecular Pharmacology: 29-63 [American Chemical Society WAshington D.C., USA].

Hungerford J. M. (1995) AOAC Official method 959.08. Paralytic shellfish poison. Official methods of Analysis, 16 edn (Arlington, Va.: AOAC International) Chapter 35.1.37.

Jellett J. F., Marks L. J. Stewart J. E., Dorey M. L. Watson-Wright W., and Lawrence J. F. (1992) Paralytic shellfish poison (saxitoxin family) bioassays: Automated endpoint determination and standardization of the in vitro tissue culture bioassay, and comparison with the standard mouse bioassay. Toxicon 30, 1143-1156.

Johnson, H. M., and Mulberry, G. (1966) Paralytic shellfish poison: serological assay by passive haemagglutination and bentonite flocculations. Nature 211, 747-748

Llewellyn L. E., Bell P. M. and Moczydlowski E. G. (1997) Phylogenetic survey of soluble saxitoxin-binding activity in pursuit of the function and molecular evolution of saxiphilin, a relative of tranferrin. Proc. R. Soc. Lond. B. 264, 891-902.

Llewellyn, L. E. and Moczydlowski E. G. (1994) Characterisation of saxitoxin binding to saxiphilin a relative of the transferrin family that displays pH-dependent ligand binding. Biochemistry 33, 12312-12322.

Llewellyn, L. E. and Doyle, J., 2000, The effect of shellfish extracts and other matrices upon the microtitre plate saxiphilin assay for paralytic shellfish poisons. Toxicon 39, 217-224.

Llewellyn, L. E., Doyle J. and Negri, A., 1998, A high throughput, microtitre plate assay for paralytic shellfish poisons using the saxitoxin specific receptor, saxiphilin. Analytical Biochemistry 261, 51-56.

Negri A. and Llewellyn L. E. (1998) Comparative analyses by HPLC and the sodium channel and saxiphilin ³H-saxitoxin receptor assays fro paralytic shellfish toxins in crustaceans and molluscs from tropical north west Australia. Toxicon, 36, 283-298.

Oshima, Y., 1995, Post-column derivatization HPLC methods for paralytic shellfish poisons. Manual on Harmful Marine Microalgae, edited by G. M. Hallegraeff, D. M. Anderson, and A. D. Cembella (Paris: UNESCO), pp 81-94.

Sommer H and Meyer K. F. (1937) Paralytic shellfish poison. Arch. Pathol 24, 560.

Useleber E., Schneider E. and Terplan G. (1991). Direct enzyme immunoassay in microtitration plate and test strip format for the detection of saxitoxin in shellfish. Lett. Appl Microbiol. 13, 275-277.

Weigele J. B. and Barchi R. L. (1978). Analysis of saxitoxin binding in isolated rate synaptosomse using a rapid filtration assay. FEBS Lett. 91, 310-314.

Van Egmond, H. P., Dekker, W. H., 1995. Worldwide regulations for mycotoxins in 1994. Natural Toxins 3, 332-336.

1. A method of detecting and/or measuring the amount of paralytic shellfish toxin (PST) present in a sample, comprising the steps of:
1) providing an isolated and purified invertebrate saxiphilin, or a fragment thereof which contains a saxitoxin binding site;
2) contacting it with the sample;
3) measuring binding of PSTs to the invertebrate saxiphilin; and
4) correlating the amount of binding with either the presence or absence of PSTs in the sample or with the PST concentration in the sample.

2. A method as claimed in claim 1 wherein the invertebrate saxiphilin, or fragment thereof, is coupled to a detectable label to provide a labelled saxiphilin.

3. A method as claimed in claim 2 wherein a predetermined amount of the labelled saxiphilin binds an immobilised PST to a predetermined extent in the absence of a PST in the sample, but to a lesser extent when a PST is present in the sample.

4. A method as claimed in claim 3 wherein the label is selected from the group consisting of fluorescent labels, chemiluminescent labels, colloidal gold, latex microbeads and enzymic labels.

5. A method as claimed in claim 4 wherein the label is selected from the group consisting of colloidal gold and coloured latex microbeads in order to provide a visual signal.

6. A method as claimed in claim 5 wherein a PST is printed to a test strip and a visual signal is produced through binding of the labelled saxiphilin thereto to form a coloured spot.

7. A method as claimed in claim 2 wherein the labelled saxiphilin is contained within and a PST is immobilised within a well of a microtitre plate, and the degree of colour formation is measured using a spectrophotometric plate reader.

8. A method as claimed in claim 7 wherein the PST is coated onto a well of the microtitre plate.

9. A method as claimed in claim 3 or claim 8 wherein the immobilised PST is saxitoxin.

10. A method as claimed in claim 1 wherein the invertebrate saxiphilin is immobilised on a solid support.

11. A method as claimed in claim 10 wherein the solid support is a test strip.

12. A method as claimed in claim 11 wherein a labelled PST binds to the invertebrate saxiphilin to a predetermined extent in the absence of a PST in the sample, but to a lesser extent when a PST is present.

13. A method as claimed in claim 12 wherein the PST is saxitoxin.

14. A method as claimed in claim 13 wherein the label is a liposome encapsulated dye with saxitoxin bound to the liposome.

15. A method as claimed in claim 14 wherein the liposome additionally has biotin bound thereto.

16. A method as claimed in claim 15 wherein a sample for analysis is carried first through an immobilised saxiphilin zone and then a liposome capture zone comprising avidin.

17. A method as claimed in claim 1 wherein binding of PSTs to the invertebrate saxiphilin is measured spectrophotometrically.

18. A method as claimed in claim 1 wherein binding of PSTs to the invertebrate saxiphilin is measured by detecting a change in mass or refractive index upon binding.

19. A method as claimed in claim 18 which employs a surface-plasmon resonance (SPR) sensor.

20. A method as claimed in any one of claims 1 to 19 wherein the invertebrate saxiphilin is centipede saxiphilin.

21. A method as claimed in claim 20 wherein the invertebrate saxiphilin is from Ethmostigmus rubripes.

22. A method of measuring the amount of paralytic shellfish toxin (PST) present in a sample, comprising the steps of:
(a) pre-treating the filters of a microtitre filtration plate with a polycation;
(b) adding to wells of the plate a known amount of a labelled saxiphilin comprising an isolated and purified invertebrate saxiphilin, or a fragment thereof which contains a saxitoxin binding site labelled with a detectable marker, and a series of dilutions of material suspected to comprise paralytic shellfish toxin;
(c) incubating the plate for a time sufficient to permit binding of any paralytic shellfish toxin present to the labelled saxiphilin;
(d) aspirating the contents of each well through the filter of the well to remove components other than labelled saxiphilin and compounds bound thereto;
(e) rinsing each well and filter to remove residual unbound compounds; and
(f) measuring the amount of labelled saxiphilin retained by the filter,
in which the degree of binding of labelled saxiphilin when compared with a control sample indicates the amount of paralytic shellfish toxin present in the sample.

23. A method as claimed in claim 22 wherein the sample comprises a buffer to maintain pH in the range 6.5 to 9.

24. A method as claimed in claim 23 wherein the sample further comprises a chloride salt, such as sodium chloride or potassium chloride, present at a concentration up to 500 mM.

25. A method as claimed in any one of claims 22 to 24 wherein the total volume present in the well is 50 to 350 μl, preferably 100 to 200 μl, more preferably 150 μl.

26. A method as claimed in any one of claims 22 to 25 wherein, in step (c), the incubation is carried out at 0 to 30° C., preferably at room temperature, for between 30 minutes and 8 hours; preferably for between 60 to 120 minutes and more preferably for 90 minutes.

27. A method as claimed in any one of claims 22 to 26 wherein, in step (e), the rinse is performed with a solution buffered at the same pH as the sample.

28. A method as claimed in any one of claims 22 to 27 wherein the invertebrate saxiphilin is centipede saxiphilin.

29. A method as claimed in claim 28 wherein the invertebrate saxiphilin is from Ethmostigmus rubripes.

30. An isolated and purified invertebrate saxiphilin coupled to a solid support.

31. An isolated and purified invertebrate saxiphilin labelled with a detectable label.

32. A kit for measuring the amount of paralytic shellfish toxin (PST) in a sample, comprising
(a) a microtitre plate;
(b) a labelled saxiphilin comprising an isolated and purified invertebrate saxiphilin, or a fragment thereof which contains a saxitoxin binding site labelled with a detectable marker;
(c) extraction buffer for extracting material to be tested an organism or tissue to be tested; and optionally
(d) a concentrating means for concentrating PSTs in the extract or for removal of contaminants that may interfere with the assay.

33. A kit as claimed in claim 32 wherein the concentrating means is a column or cartridge comprising a solid support material coupled to an isolated and purified saxiphilin.

34. A device for measuring the amount of paralytic shellfish toxin (PST) present in a sample, comprising:
an immobilised invertebrate saxiphilin, or a fragment thereof which contains a saxitoxin binding site;
means for introducing a sample to the immobilised invertebrate saxiphilin, or fragment thereof;
means for measuring binding of PSTs contained in the sample to the immobilised invertebrate saxiphilin, or fragment thereof; and
means for correlating the amount of binding with either the presence or absence of PSTs or with PST concentration in the sample.

35. A device as claimed in claim 34 wherein the immobilised invertebrate saxiphilin is centipede saxiphilin.

36. A device as claimed in claim 35 wherein the immobilised invertebrate saxiphilin is from Ethmostigmus rubripes.

37. A device as claimed in any one of claims 34 to 36 comprising a diagnostic test strip including an immobilised saxiphilin zone.

38. A device as claimed in claim 37 further comprising an avidin zone positioned further from the end of the test strip at which a sample is introduced than the immobilised saxiphilin zone.

39. A diagnostic test strip comprising a wick including a zone where saxiphilin is immobilised thereon and a zone further from the end of the wick within which avidin is bound.

40. A kit comprising a diagnostic test strip as defined in claim 39, saxitoxin- and biotin-tagged liposome encapsulated-dye and, optionally, a buffer solution.

41. A biosensor for measuring the amount of paralytic shellfish toxin (PST) present in a sample, comprising:
an immobilised invertebrate saxiphilin, or a fragment thereof which contains a saxitoxin binding site;
means for introducing a sample to the immobilised invertebrate saxiphilin, or fragment thereof;
means for measuring binding of PSTs contained in the sample to the immobilised invertebrate saxiphilin, or fragment thereof; and
means for translating the binding event into an electronic signal and correlating the amount of binding with either the presence or absence of PSTs or with PST concentration in the sample.

42. A biosensor as claimed in claim 41 wherein the means for translating the binding event into an electronic signal involved detection of the change of mass of the protein upon binding.

43. A biosensor as claimed in claim 42 wherein the immobilised invertebrate saxiphilin is a fragment of the saxiphilin protein containing the saxitoxin binding site is used in order to maximise the change in mass upon binding.

44. A biosensor as claimed in any one of claims 41 to 43 wherein the immobilised invertebrate saxiphilin is centipede saxiphilin.

45. A biosensor as claimed in claim 44 wherein the immobilised invertebrate saxiphilin is from Ethmostigmus rubripes.

46. A device for measuring the amount of paralytic shellfish toxin (PST) present in a sample, comprising:
an immobilised PST;
means for introducing a sample to said immobilised PST;
means for introducing a predetermined amount of an isolated and purified invertebrate saxiphilin to the sample;
means for measuring binding of the invertebrate saxiphilin introduced to said immobilised PST; and
means for correlating competition for binding between the immobilised PST and any PST contained in the sample with PST concentration in the sample.

47. A device as claimed in claim 46 wherein the PST is saxitoxin.

48. A device as claimed in claim 47 wherein the saxitoxin is printed onto a diagnostic test strip.

49. A diagnostic test strip to which saxitoxin is printed.

50. A biosensor for measuring the amount of paralytic shellfish toxin (PST) present in a sample, comprising:
an immobilised PST;
means for introducing a sample to said immobilised PST;
means for introducing a predetermined amount of an isolated and purified invertebrate saxiphilin to the sample;
means for measuring binding of the introduced invertebrate saxiphilin to said immobilised PST; and
means for translating the binding event into an electronic signal and correlating the amount of binding with either the presence or absence of PSTs or with PST concentration in the sample.

51. A biosensor as claimed in any one of claims 41 to 43 wherein the immobilised PST is saxitoxin.

52. A method of isolating and purifying an invertebrate saxiphilin, comprising the steps of:
(a) homogenising individuals of a saxiphilin-producing invertebrate species in a physiological buffer comprising protease inhibitors;
(b) subjecting the homogenate to low-speed centrifugation to remove cell debris;
(c) subjecting the supernatant from step (b) to high-speed centrifugation;
(d) precipitating crude saxiphilin from the supernatant by exposure to ammonium sulphate;
(e) redissolving the precipitate at pH 5.0-6.5 and centrifuging to remove non-saxiphilin molecules;
(f) exposing the supernatant from (e) to a cationic matrix which binds saxiphilin such as a glass fibre-polyethylene imine (PEI) support matrix; and
(g) eluting bound material from the matrix under high salt conditions, whereby an isolated and purified invertebrate saxiphilin is produced.

53. A method as claimed in claim 52 wherein the saxiphilin is eluted by NaCl or KCl at a concentration from 600 mM to saturation, in buffer at pH 5-9.

54. A method as claimed in claim 52 wherein the saxiphilin is precipitated by exposure to 40-60% ammonium sulphate.

55. A method as claimed in any one of claims 52 to 54 wherein the invertebrate is a centipede.

56. A method as claimed in claim 55 wherein the centipede is Ethmostigmus rubripes.

57. An invertebrate saxiphilin when prepared by the process of any one of claims 52 to 56.

58. A method for the concentration, purification and/or extraction of paralytic shellfish toxins (PSTs), comprising the steps of:
providing an immobilised invertebrate saxiphilin, or a fragment thereof which contains a saxitoxin binding site;
contacting a sample suspected of containing a PST with the immobilised invertebrate saxiphilin for a sufficient time for the PST to bind said immobilised invertebrate saxiphilin; and
optionally, eluting the bound PST from the immobilised invertebrate saxiphilin.

59. A method as claimed in claim 60 wherein the immobilised invertebrate saxiphilin is centipede saxiphilin.

60. A method as claimed in claim 61 wherein the immobilised invertebrate saxiphilin is from Ethmostigmus rubripes.

61. A method as claimed in any one of claims 58 to 60 wherein the method is used to detoxify shellfish.

62. A method as claimed in any one of claims 50 to 60 wherein PSTs are extracted from drinking water.

63. Use of an isolated and purified invertebrate saxiphilin in the preparation of affinity materials for concentration, purification and/or extraction of paralytic shellfish toxins.

64. An affinity material for concentration, purification and/or extraction of paralytic shellfish toxins, comprising an isolated and purified invertebrate saxiphilin, or fragment thereof which contains a saxitoxin binding site coupled to a solid support.

65. An affinity material as claimed in claim 64 wherein the solid support is packed into a cartridge or column.

66. An affinity material as claimed in either one of claims 64 or 65 wherein the solid support is selected from the group consisting of azolactone coupling matrices, cyanogen bromide-activated matrices; epoxy activated matrices; glutaraldehyde-activated silica; carboxymethylcellulose hydrazide; polyacrylamide hydrazide and oxirane acrylic beads.
